(19) United States
(12) Patent Application Publication
(10) Pub. No.: US 2007/0105898 A1
(43) Pub. Date: May 10, 2007

(54) Process for the production of cilostazol

(75) Inventors: YuanQiang Li, Brantford (CA); Zhi-Xian Wang, Brantford (CA); Stephen E. Horne, Burlington (CA); Bhaskar Reddy Guntoori, Brantford (CA)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, CA

(73) Assignee: Apotex Pharmachem Inc.

(21) Appl. No.: 11/269,541

(22) Filed: Nov. 9, 2005

(51) Int. Cl. A61K 31/4709; C07D 403/02

(52) U.S. Cl. 514/312; 546/158

(57) ABSTRACT

A process for the preparation of cilostazol of formula I from 6-hydroxy-3,4-dihydroquinolinone of formula II and 1-cyclohexyl-5-(4-halobutyl)-tetrazole of formula III, 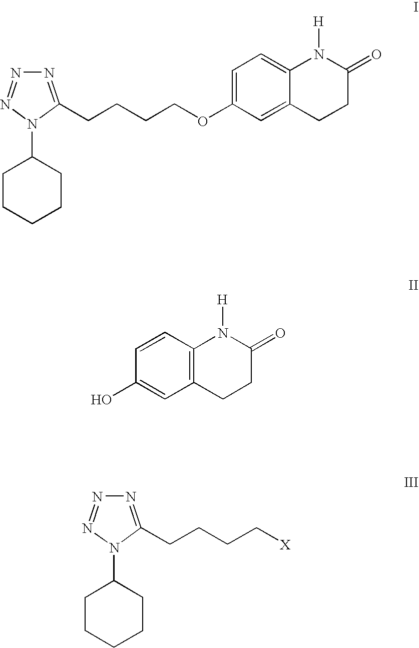 wherein X is a halogen atom such as Cl, Br, and I, that includes combining compounds II, III, a water-miscible organic solvent, a water-soluble base and water. The cilostazol can then be separated from the reaction mixture and dissolved in a solvent A. The resulting cilostazol solution is mixed with a solvent B to precipitate cilostazol particles of defined particle size range, milling the precipitate if desired, and filtering and drying the product.

### FIELD OF THE INVENTION

The present invention relates to processes for producing cilostazol.

### BACKGROUND OF THE INVENTION

The present invention pertains to processes for producing 6-[4-(1-cyclohexyl-1H-tetrazol-5-yl)butoxy]-3,4-dihydro-2(1H)-quinolinone of formula (I), which is also known by the generic name cilostazol. Cilostazol inhibits cell platelet aggregation and is used to treat patients with intermittent claudication.

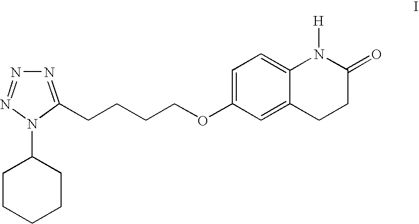

A synthetic preparation of cilostazol is disclosed in U.S. Pat. No. 4,277,479, whereby a 6-hydroxy-3,4-dihydroquinolinone of formula II is alkylated with a 1-cyclohexyl-5-(4-halobutyl)-tetrazole of formula III, wherein X is a halogen atom such as Cl, Br, and I. It is recommended to use an equimolar or excess amount of up to two molar equivalents of the compound III. 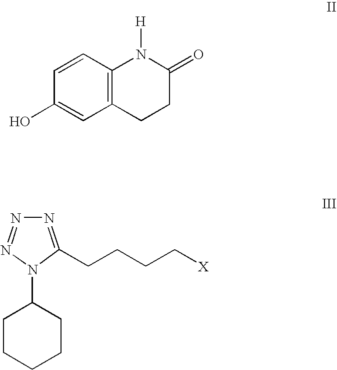
U.S. Pat. No. 4,277,479 patent states that the alkylation may be conducted neat or in a solvent. Suitable solvents are said to be methanol, ethanol, propanol, butanol, ethylene glycol, dimethyl ether, tetrahydrofuran, dioxane, monoglyme, diglyme, acetone, methyl ethyl ketone, benzene, toluene, xylene, methyl acetate, ethyl aceatate, N,N-dimethylformamide, dimethyl sulfoxide and hexamethylphosphorus triamide (HMPT). According to Examples 4 and 26 of the U.S. Pat. No. 4,277,479 patent, cilostazol was prepared using 1,8-diazabicyclo[5,4.0]undec-7-ene (DBU) as base and ethanol as solvent.

In Nishi, T. et al., Chem. Pharm. Bull. 1983, 31, 1151-57, a preparation of cilostazol is described wherein II is reacted with 1.2 molar equivalents of III in isopropanol with potassium hydroxide as a base. Cilostazol was obtained in 74% yield.

U.S. Pat. No. 6,515,128 and JP 2001213877 describe processes for preparing cilostazol in heterogeneous or biphasic systems in the presence of a phase transfer catalyst. U.S. Pat. No. 6,515,128 also discloses a homogeneous process where the reaction mixture has to be anhydrous. This was achieved using molecular sieves before compound III is added and the process was relatively low yielding. U.S. Pat. No. 6,630,590 discloses a process of preparing cilostazol in a single aqueous phase in the presence of a phase transfer catalyst and sodium sulfite and the reaction was carried out by circulating the reaction mixture with a continuous disperser.

One reason for using an excess of compound III as was done by Nishi et al. and recommended by U.S. Pat. No. 4,277,479 is that it is unstable to some bases. U.S. Pat. No. 6,515,128 discloses that when exposed to an alkali metal hydroxide in water for a sufficient period, compound III, which is expensive, undergoes elimination and cyclization to yield byproducts IV and V. It is also notable that the yields disclosed in the U.S. Pat. No. 4,277,479 and Nishi's article were relatively low, and the process involved complicated work-up procedures such as column chromatography.

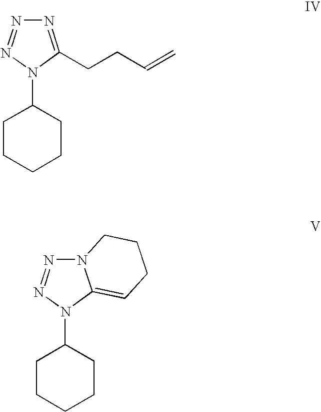

The later patents (U.S. Pat. No. 6,515,128; JP 2001213877; U.S. Pat. No. 6,630,590) partially addressed some of the problems in the original process disclosed in the U.S. Pat. No. 4,277,479 by using phase transfer catalyst and reaction promoters such as sodium sulfate. However, the processes disclosed in those patents usually involved a heterogeneous, or biphasic mixture, which increases process complexity and may cause problems on scale-up. Also, new chemicals such as phase transfer catalysts and reaction promoters were used in those processes and they will introduce extra cost to the production of cilostazol and generate more chemical waste. The process disclosed in U.S. Pat. No. 6,630,590 requires circulating the reaction mixture using a continuous disperser, which is not conventional equipment in a commercial plant.

It is also sometime the case, that in order to improve the solubility and bioavailability of a compound, having specific particles sizes can results in the increase of these two properties. Mechanical methods of obtaining particle sizes of compound are known in the art, for example, processes such as fine-milling and micronization. However, such process are unsuitable and difficult to achieve for very small particles such as those having a d(0.9)≦40 microns. Fine-milling and micronization to achieve such small particle sizes require special equipment and results in a significant product loss.

Therefore, it would be highly desirable to find a simple, low cost and highly efficient alternate process of producing cilostazol overcoming the deficiencies of the prior art.

### SUMMARY OF THE INVENTION

In one aspect of the invention, there is provided for an improved process for producing cilostazol (I) by alkylating the phenol group of compound II at the δ carbon of a 1-cyclohexyl-5-(4-halobutyl)-tetrazole (III), wherein X is a halogen atom such as Cl, Br, and I.

In another aspect of the invention, there is provided for a highly efficient homogeneous process of producing cilostazol via the reaction of compounds II and III in a water miscible organic solvent in the presence of a water-soluble base and water. The cilostazol product is produced in high yield and purity.

In yet another aspect of the invention, there is provided for a process to produce cilostazol particles of defined particle size range. In this context, the term “defined particle size range” means that about 90% of the particles have a diameter equal to or less than about 40 micrometres (d(0.9)≦40 micrometres) and about 50% of the particles have a diameter equal to or less than about 10 micrometres (d(0.5)≦10 micrometres).

In still yet another aspect of the invention, there is provided for the precipitation of ciloztazol in a process for the preparation of cilostazol particles of a defined particle size range comprising of dissolving cilostazol in a first solvent in which cilostazol is soluble, mixing with the first solvent, a second solvent in which cilostazol has lower solubility wherein the second solvent is different from the first solvent and whereby said cilostazol precipitates; and filtering and drying of the resulting cilostazol particles.

In a further aspect of the invention, there is provided for a process for the preparation of cilostazol particles suitable for use in a pharmaceutical composition for oral administration.

In another aspect of the invention, there is provided for a homogeneous process that is suitable for large scale production. The process does not require an excess amount of expensive compound III nor extra reagents such as phase transfer catalysts and reaction promoters. The process involves a reaction that can be carried out in the presence of water, therefore a dehydrating reagent is not needed. Also, the solvent(s) and base(s) used in the process are inexpensive and commercially available and the process does not require special manufacturing equipment.

### DETAILED DESCRIPTION OF PREFERRED EMBODIMENTS

An improved process for the production of cilostazol (I) by alkylating the phenol group of compound II at the δ carbon of 1-cyclohexyl-5-(4-halobutyl)tetrazole (III), wherein X is a halogen atom such as Cl, Br, and I is exemplified. The transformation itself, depicted in Scheme 1, is known. The present invention improves upon processes previously reported to perform the chemical transformation depicted in Scheme 1, which results in a greater conversion of the starting materials II and III to cilostazol by employing a highly efficient homogeneous process. The cilostazol prepared can be further subjected to a dissolution-precipitation process to prepare cilostazol particles having reduced particle size.

In one embodiment of the invention, the process comprises the following: combining compounds II, III, a water-miscible organic solvent, a water-soluble base and water; heating the reaction mixture; and separating cilostazol from the reaction mixture. The cilostazol can also be further formed into particles of defined particle size by dissolving cilostazol in a first solvent in which cilostazol is soluble; mixing the cilostazol solution and a second solvent to precipitate cilostazol particles of defined size range (the second solvent is a solvent in which cilostazol has lower solubility); milling, if desired; and filtering and drying the product.

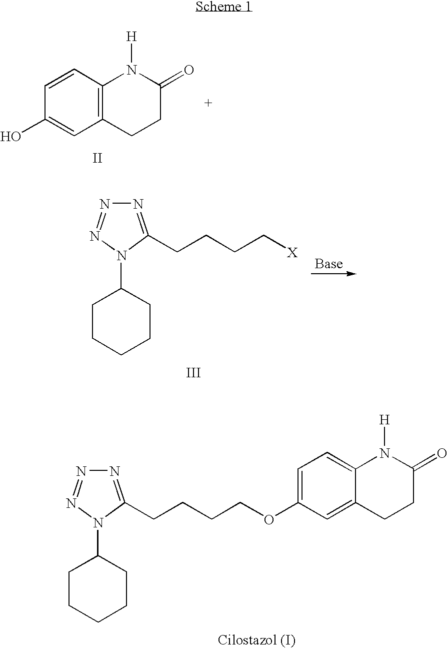

The halogen atom of 1-cyclohexyl-5-(4-halobutyl)tetrazole (X in formula III) may be chlorine, bromine or iodine, preferably chlorine. Although the compound III may be used in any amount desired, it is most desirable to use a stoichiometric amount of compound III or less relative to compound II, more preferably between 0.9 and 1.0 molar equivalents.

The suitable water miscible solvents include C1 to C8 alcohols such as butanols, propanols, ethanol and methanol; N,N-dialkylamides such as N,N-dimethylformamide, N,N-dimethylacetamide and N-methyl-2-pyrrolidinone; cyclic and acyclic alkyl sulfoxides and sulfones such as dimethyl sulfoxide and sulfolane; and alkylphosphorylamides such as hexamethylphosphoramide and hexamethylphosphorus triamide. The most preferred solvents are N,N-dialkylamides such as N,N-dimethylformamide, N,N-dimethylacetamide and N-methyl-2-pyrrolidinone. The amount of solvent can range from 0.5 volumes to 20 volumes relative to compound II, preferably from 1 volume to 5 volumes.

Suitable water soluble bases include alkali metal hydroxides such as sodium hydroxide, potassium hydroxide, lithium hydroxide; alkali metal carbonates such as sodium carbonate, potassium carbonate and lithium carbonate; alkali metal bicarbonates such as sodium bicarbonate, potassium bicarbonate and lithium bicarbonate; and alkali metal alkylates such as sodium methoxide, sodium ethoxide, sodium t-butoxide, and potassium t-butoxide. The most preferred bases are alkali metal hydroxides such as sodium hydroxide, potassium hydroxide, and lithium hydroxide. The amount of base ranges from 0.9 to 1.5 equivalents relative to compound II, more preferably 0.9 to 1.1 equivalents.

The water can be premixed with the base to form a solution of the base and added to the reaction mixture or, optionally, directly added into the reaction mixture. The ratio of water to organic solvent may range from 1:100 to 2:1 volume by volume (v/v), preferably from 1:5 to 1:1 (v/v).

The mixture of compounds II and III in a water miscible organic solvent in the presence of a water-soluble base and water can be heated for a period of time sufficient for reaction completion and then separating the product cilostazol from the reaction mixture. The compounds II, III, organic solvent, water-soluble base and water may be added in any order desired and at any rate desired. In one preferred embodiment, compound II, organic solvent, base and water are mixed. Thereafter, compound III is added to the mixture and it is heated to complete the reaction.

The reaction temperature may range from 50° C. to 180° C., preferably between 50° C. and 120° C., more preferably between 70° C. and 100° C.

Although the cilostazol product may be separated from the reaction medium by any separation techniques such as crystallization, liquid-liquid extraction, and column chromatography, for instance, the latter being used in the process of Nishi et al. (Chem. Pharm. Bull. 1983, 31, 1151-57), it is desirable on large scale to directly isolate the product from the reaction mixture through crystallization. The cilostazol prepared according to the teachings of the present invention can be selectively precipitated from the reaction mixture in high purity, by the addition of an anti-solvent.

The cilostazol can be first dissolved in any suitable first solvent in which cilostazole is soluble. These include C1 to C8 alcohols such as methanol, ethanol, propanols and butanols; ayclic and cyclic N,N-dialkylamides such as N,N-dimethylformamide, N,N-dimethylacetamide and 1-methyl-2-pyrrolidinone; cyclic and acyclic alkyl sulfoxides and sulfones such as dimethyl sulfoxide and sulfolane; haloalkanes such as dichloromethane, dichloroethane, and chloroform; C3 to C8 alkyl ketones such as acetone, methyl ethyl ketone and methyl isobutyl ketone; C2 to C6 alkyl nitrites such as acetonitrile; C1 to C7 alkylcarboxylic acids such as acetic acid, propionic acid and butyric acid; and mixtures thereof. The more preferred solvents are methanol, N,N-dimethylformamide, N,N-dimethylacetamide, 1-methyl-2-pyrrolodinone, acetone, methyl isobutyl ketone, methyl ethyl ketone, acetic acid and mixtures thereof.

The dissolution may be conducted at any temperature if desired, preferably 50° C. to 150° C.

To this cilostazol solution, there can be mixed in a second solvent, the anti-solvent, which causes the cilostazol to precipitate. The anti-solvent being a solvent in which cilostazol has lower solubility to cause precipitation. This second solvent is necessarily different from the first solvent and can include water, C5 to C12 hydrocarbons including toluene, xylenes, heptanes and hexanes; ethers such as C3 to C10 esters including methyl acetate, ethyl acetate, isopropyl acetate, and butyl acetate, nitriles, including C2 to C5 nitriles, including acetonitrile, ketones such as C3 to C8 alkyl ketones, including acetone, methyl isobutyl ketone and methyl ethyl ketone, and C1 to C8 alkyl alcohols such as methanol, ethanol, propanols and butanols,; and mixtures thereof. The most preferred anti-solvents are water, ethyl acetate, isopropyl acetate, methyl isobutyl ketone and mixtures thereof.

The ratio of the first solvent to the second solvent can range from 2:1 to 1:100 (v/v), and preferably 1:1 to 1:20 (v/v). The precipitation may be caused by the addition of the cilostazol solution (consisting of the cilostazol and the cilostazol solublizing solvent) into the anti-solvent or the addition of the anti-solvent into the cilostazol solution at any rate desired. In one preferred embodiment, the cilostazol solution is added slowly into the anti-solvent with stirring. The resulting suspension can be further subjected to milling if desired. The suspension is filtered and the solid is dried to give cilostazol particles of reduced particle size. The cilostazol particles so obtained according to the process have a particle size distribution of d(0.9)≦40 microns and d(0.5)≦10 micrometres.

The following non-limiting examples further illustrate the manner of carrying out the inventive process described herein.

### EXAMPLE 1

To a mixture of 6-hydroxy-3,4-dihydroquinolinone (II) (75.0 g, 0.46 mol) in 1-methyl-2-pyrrolidinone (150 mL) was added 50% aqueous sodium hydroxide solution (36.8 g, 0.46 mol) and water (75 mL). The mixture was stirred and heated to 50-60° C. to give a solution. 1-Cyclohexyl-5-(4-chlorobutyl)-tetrazole (III, X=Cl) (100.5 g, 0.414 mol) was added and the resulting mixture was heated to 85-95° C. and stirred for 3 hrs. The reaction was determined to be complete by TLC. After being cooled to 50-60° C., isopropyl acetate (225 mL) and water (450 mL) were added and the reaction mixture was heated to reflux (about 80° C.). The mixture was cooled to 0-5° C. and stirred for 2 hrs. The resulting suspension was filtered, rinsed with isopropyl acetate (150 mL), water/1-methyl-2-pyrrolidinone (7/2 v/v, 150 mL) and water (150 mL) and dried to give 130.66 g (yield. 85.4%) of cilostazol. HPLC purity: 99.53%.

### EXAMPLE 2

A hot solution (70-75° C.) of cilostazol (23 g) in 1-methyl-2-pyrrolidinone (69 mL) was slowly added over a period of 5-10 min into water (345 mL) at 0-5° C. with stirring. The suspension was stirred at 0-5° C. for 1-2 hrs, filtered and rinsed with water (40 mL) and methanol (20 mL). The solid was dried under vacuum at 55-60° C. to give 22.1 g (yield 96%) cilostazol as a white powder. Particle size distribution: d(0.1)=0.71 micrometres, d(0.5)=5.94 mircrometres, d(0.9)=30.59 micrometres.

### EXAMPLE 3

A hot solution (70-75° C.) of cilostazol (20 g) in 1-methyl-2-pyrrolidinone (40 mL) was slowly added over a period of 5-10 min into toluene (250 mL) at 0-5° C. with stirring. The suspension was stirred at 0-5° C. for 1-2 hrs, filtered and rinsed with toluene (100 mL). The solid was dried under vacuum at 55-60° C. to give 18.2 g (yield 91%) cilostazol as a white powder. Particle size distribution: d(0.1)=0.80 micrometres, d(0.5)=3.90 mircrometres, d(0.9)=25.56 micrometres.

While the foregoing provides a detailed description of the preferred embodiments of the invention, it is to be understood that the descriptions are illustrative only of the principles of the invention and not limiting. Furthermore, as many changes can be made to the invention without departing from the scope of the invention, it is intended that all material contained herein be interpreted as illustrative of the invention and not in a limiting sense.

1. A process for the preparation of cilostazol of formula I comprising reacting 6-hydroxy-3,4-dihydroquinolinone of formula II and 1-cyclohexyl-5-(4-halobutyl)tetrazole of formula III, 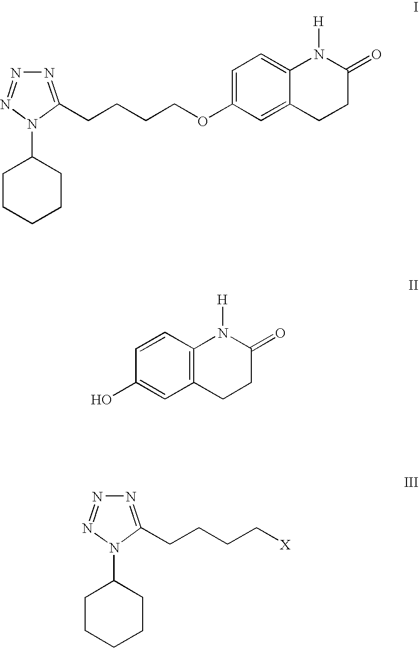
wherein X is a halogen atom, in a solvent mixture, said solvent mixture comprising:
(a) a water-miscible organic solvent,
(b) a water-soluble base, and
(c) water.

2. A process according to claim 2 wherein said water miscible-organic solvent is selected from the group consisting of butanols, propanols, ethanol, methanol, N,N-dimethylformamide, N,N-dimethylacetamide, N-methyl-2-pyrrolidinone, dimethyl sulfoxide, sulfolane, hexamethylphosphoramide and hexamethylphosphorus triamide.

3. A process according to claim 1 wherein said water-soluble base is selected from the group consisting of alkali metal hydroxides, alkali metal carbonates, alkali metal bicarbonates, and alkali metal alkylates.

4. A process according to claim 1 wherein said water-soluble base is selected from sodium hydroxide, potassium hydroxide, and lithium hydroxide.

5. A process according to claim 1 wherein said compound of formula III is present in an amount of between 0.9 and 1.0 molar equivalents to said compound of formula II.

6. A process according to claim 1 wherein said water-miscible organic solvent is present in an amount of between 1 and 5 (volume by volume) to said compound of formula II.

7. A process according to claim 1 wherein the ratio of said water to said water-miscible organic solvent is between 1:100 and 2:1 (volume by volume).

8. A process according to claim 1 wherein said water-soluble base is present in an amount of between 0.9 and 1.5 equivalents to said compound of formula II.

9. A process according to claim 1 wherein the reaction proceeds at a temperature between 50° C. and 120° C.

10. A process according to claim 1 further comprising dissolving said cilostazol in a first solvent in which said cilostazol is soluble and mixing with said first solvent, a second solvent in which said cilostazol has lower solubility wherein said second solvent is different from said first solvent, whereby said cilostazol precipitates in particle form having a defined particle size range.

11. A process according to claim 10 wherein said first solvent is selected from a group consisting of methanol, ethanol, propanols, butanols, N,N-dimethylformamide, N,N-dimethylacetamide, 1-methyl-2-pyrrolidinone, dimethyl sulfoxide, sulfolane, dichloromethane, dichloroethanes, chloroform, acetone, methyl ethyl ketone, methyl isobutyl ketone, acetonitrile, acetic acid, propionic acid, butyric acid and mixtures thereof.

12. A process according to claim 10 wherein said second solvent is selected from a group consisting of water, methyl ethyl ketone, methyl isobutyl ketone, propanols, butanols, toluene, xylenes, heptanes, hexanes, methyl acetate, ethyl acetate, isopropyl acetate, butyl acetate, acetonitrile and mixtures thereof.

13. A process for the preparation of cilostazol particles of a defined particle size range comprising of:
(a) dissolving cilostazol in a first solvent in which cilostazol is soluble;
(b) mixing with said first solvent, a second solvent in which cilostazol has low solubility wherein said second solvent is different from said first solvent and whereby said cilostazol precipitates; and
(c) filtering and drying said cilostazol particles.

14. A process according to claim 13 wherein said first solvent is selected from the group consisting of methanol, ethanol, propanols, butanols, N,N-dimethylformamide, N,N-dimethylacetamide, 1-methyl-2-pyrrolidinone, dimethyl sulfoxide, sulfolane, dichloromethane, dichloroethanes, chloroform, acetone, methyl ethyl ketone, methyl isobutyl ketone, acetonitrile, acetic acid, propionic acid, butyric acid and mixtures thereof.

15. A process according to claim 13 wherein said second solvent is selected from a group consisting of water, methyl ethyl ketone, methyl isobutyl ketone, propanols, butanols, toluene, xylenes, heptanes, hexanes, methyl acetate, ethyl acetate, isopropyl acetate, butyl acetate, acetonitrile and mixtures thereof.
